(19) United States
(12) Patent Application Publication
(10) Pub. No.: US 2025/0316374 A1
(43) Pub. Date: Oct. 9, 2025

(54) REMOTELY INTERFACED MEDICAL SCREENING ASSEMBLY

(71) Applicant: Yasir Jassam, Mission Viejo, CA (US)

(72) Inventor: Yasir Jassam, Mission Viejo, CA (US)

(21) Appl. No.: 18/629,381

(22) Filed: Apr. 8, 2024

(51) Int. Cl. G16H 40/67; G16H 80/00

(52) U.S. Cl. CPC G16H 40/67; G16H 80/00

(57) ABSTRACT

A remotely interfaced medical screening assembly for remotely screening and diagnosing a user includes a plurality of garments, a relay module, and a user interface device. Each garment substantially covers a respective body part of the user and is equipped with one or more sensors and stimulators, each of which can assess or elicit a biological parameter or biological response, respectively. Connectors are operationally engaged to the relay module and can be selectively mutually attached to operationally engage the sensors and the stimulators to the relay module. The user interface device is in wireless communication with the relay module and a healthcare provider's electronic device so that commands, biological parameters, and biological responses are communicable between the relay module and the healthcare provider's electronic device, allowing the healthcare provider to evaluate the user and to formulate a diagnosis.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical screening assembly and more particularly pertains to a new medical screening assembly for remotely screening and diagnosing a user. The present invention discloses a medical screening assembly that allows a healthcare provider to remotely evaluate a user via sensors and stimulators that are incorporated into a plurality of garments positioned on the user.

### (2) Description of Related Art Including Information Disclosed Under 37 CFR 1.97 and 1.98

The prior art relates to medical screening assemblies, which may comprise garments with integrals sensors, virtual display headsets, and headsets for ophthalmic examinations. What is lacking in the prior art is a medical screening assembly comprising a plurality of garments, which, upon donning, substantially cover a body of a user, and which contain a sensors and stimulators for reading biological parameters and eliciting biological responses, respectively. Further lacking in the prior art is such a medical screening assembly including a headset to enable virtual visitation with a healthcare provider, assessments of eye movement range and direction, pupil size, cognition testing, and rehabilitation exercises.

### BRIEF SUMMARY OF THE INVENTION

An embodiment of the disclosure meets the needs presented above by generally comprising a plurality of garments, a relay module, and a user interface device. Each garment is configured to be donned by the user so that the garment substantially covers a respective body part of the user. Each sensor of a plurality of sensors and each stimulator of a plurality of stimulators is attached to a respective garment. Each sensor is configured to assess a respective biological parameter of the user so that the plurality of sensors is configured to assess a variety of biological parameters of the user. Each stimulator is configured to elicit a respective biological response from the user so that the plurality of stimulators is configured to elicit a variety of biological responses from the user.

The relay module comprises a relay transceiver and is attached to a respective one of the garments. A plurality of connectors is attached to the plurality of garments, with respective connectors being operationally engaged to the relay module. The connectors are selectively mutually attachable, upon donning of the garments, so that the sensors and the stimulators are operationally engaged to the relay module. The user interface device is in wireless communication with the relay module and a healthcare provider's electronic device. The user interface device thus is configured to communicate commands, biological parameters, and biological responses between the relay module and the healthcare provider's electronic device, allowing the healthcare provider to evaluate the user and to formulate a diagnosis.

Another embodiment of the disclosure includes a method for remotely screening a user for a medical condition. The method comprises a provision step, which entails providing a remotely interfaced medical screening assembly, according to the disclosure above. Preparatory step of the method are donning the garments, mutually attaching the connectors so that the sensors and the stimulators are operationally engaged to the relay module, and initiating wireless communication between the user interface device and the healthcare provider's electronic device. Use steps of the method are allowing the sensors to assess the biological parameters of the user, allowing the stimulators to elicit the biological responses from the user, and following any commands communicated by the healthcare provider.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an in-use view of a remotely interfaced medical screening assembly according to an embodiment of the disclosure.

FIG. 2 is a front view of an embodiment of the disclosure.

FIG. 3 is a front view of an embodiment of the disclosure.

FIG. 4 is an isometric perspective view of an embodiment of the disclosure.

FIG. 5 is a front view of an embodiment of the disclosure.

FIG. 6 is a front view of an embodiment of the disclosure.

FIG. 7 is a block diagram of an embodiment of the disclosure.

FIG. 8 is a flow diagram for a method utilizing an embodiment of the disclosure.

### DETAILED DESCRIPTION OF THE INVENTION

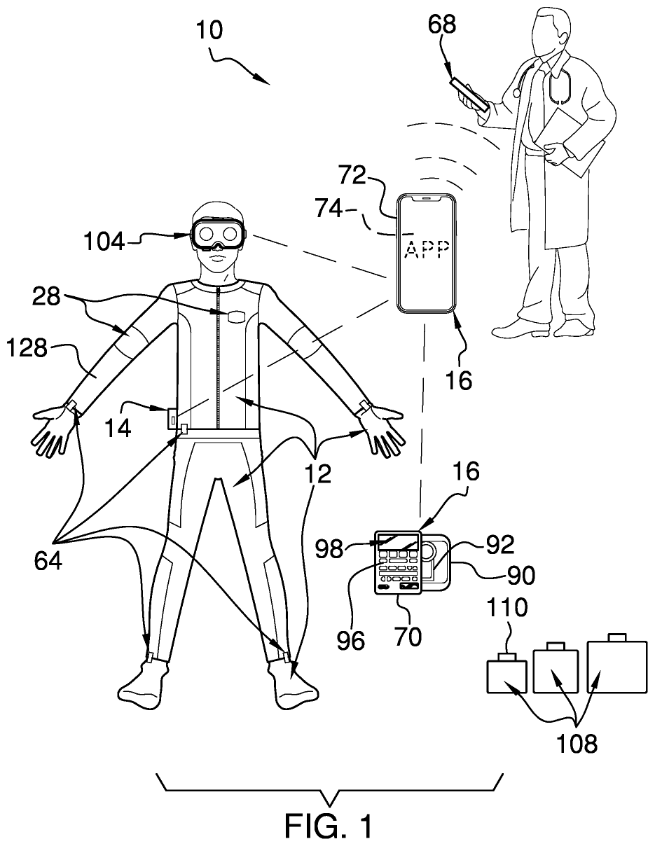

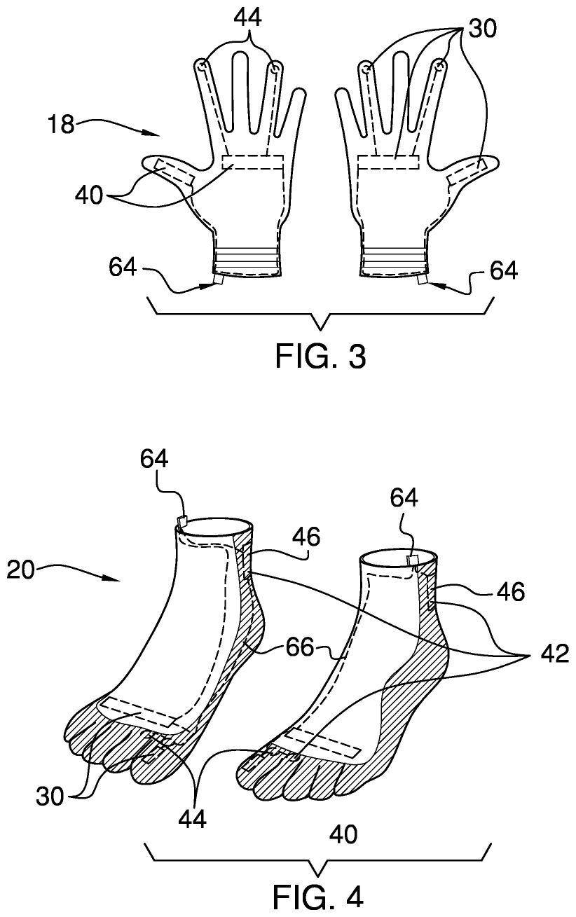

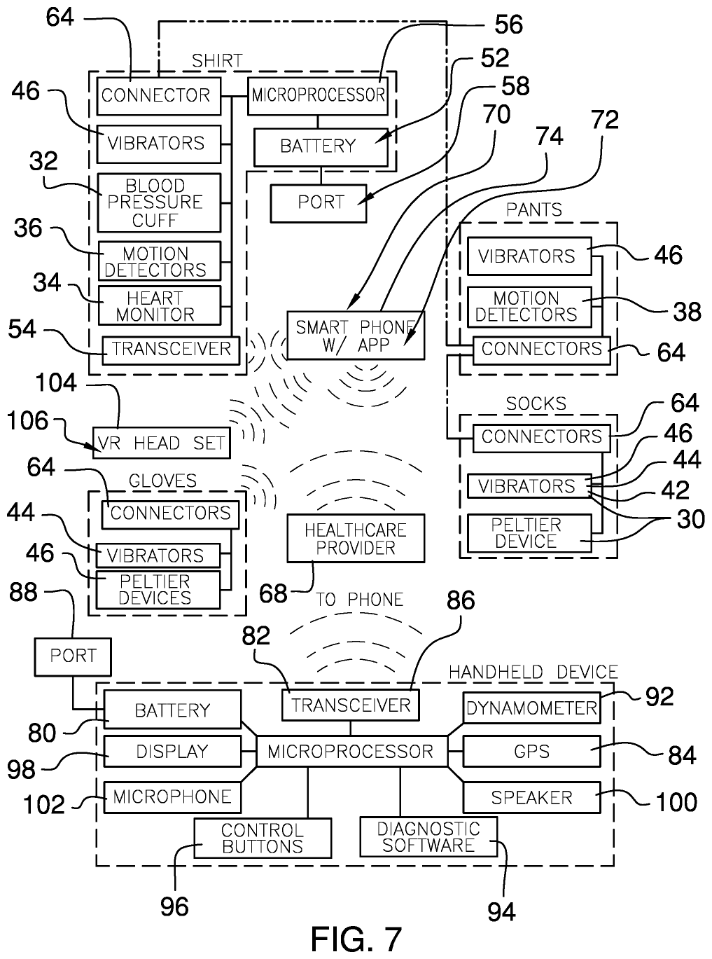

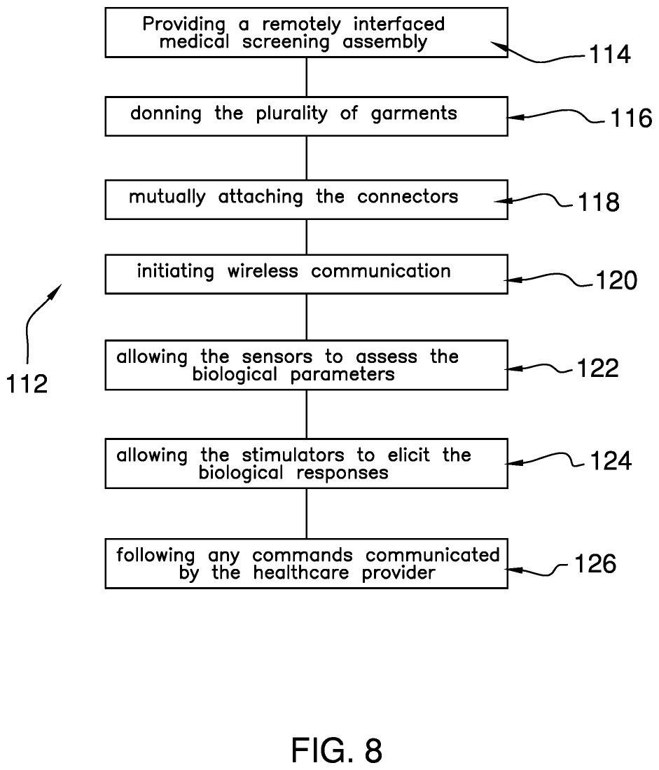

With reference now to the drawings, and in particular to FIGS. 1 through 7 thereof, a new medical screening assembly embodying the principles and concepts of an embodiment of the disclosure and generally designated by the reference numeral 10 will be described.

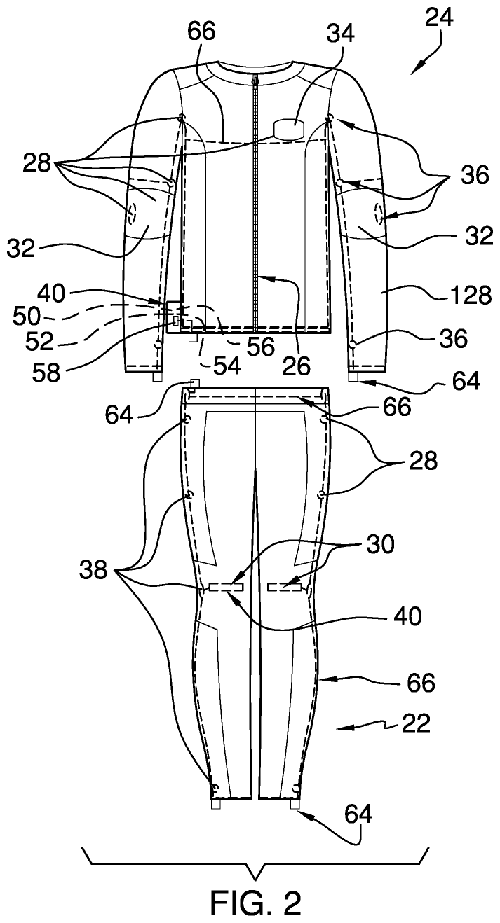

As best illustrated in FIGS. 1 through 7, the remotely interfaced medical screening assembly 10 generally comprises a plurality of garments 12, a relay module 14, and a user interface device 16. Each garment 12 is configured to be donned by the user so that the garment 12 substantially covers a respective body part of the user. As shown in FIG. 1, the plurality of garments 12 comprises a pair of gloves 18, a pair of socks 20, pants 22, and a long sleeved shirt 24. As shown in FIG. 2, the long sleeved shirt 24 comprises a zipper 26 to facilitate donning of the long sleeved shirt 24.

Each sensor 28 of a plurality of sensors 28 and each stimulator 30 of a plurality of stimulators 30 is attached to a respective garment 12. Each sensor 28 is configured to assess a respective biological parameter of the user so that the plurality of sensors 28 is configured to assess a variety of biological parameters of the user. The plurality of sensors 28 comprises a blood pressure cuff 32, which is attached to a respective sleeve 128 of the long sleeved shirt 24 and which is configured to measure a blood pressure of the user. Another blood pressure cuff 32 may be attached to the other sleeve 128 of the long sleeved shirt 24, as shown in FIG. 2.

The plurality of sensors 28 also comprises a heart monitoring device 34, which is attached to the long sleeved shirt 24 and which is configured to monitor cardiac events. Such devices for monitoring cardiac events are well known to those skilled in the art of heart monitors and include products manufactured by iRYTHEM, Medtronic, and the like.

The plurality of sensors 28 also comprises a plurality of first motion sensors 36 and a plurality of second motion sensors 38. The first motion sensors 36 are attached to the long sleeved shirt 24 so that they are positioned proximate to each shoulder, each elbow, and each wrist of the user upon donning of the long sleeved shirt 24. The first motion sensors 36 are configured to assess range and direction of movement for upper limbs of the user. The second motion sensors 38 are attached to the pants 22 so that they are positioned proximate to each hip, each knee, and each ankle of the user upon donning of the pants 22. The second motion sensors 38 are configured to assess range and direction of movement for lower limbs of the user. The present invention also anticipates additional motion detecting sensors being attached to the gloves 18 and to the socks 20.

Each stimulator 30 is configured to elicit a respective biological response from the user so that the plurality of stimulators 30 is configured to elicit a variety of biological responses from the user. The plurality of stimulators 30 comprises a plurality of Peltier coolers 40 and a plurality of vibrators 42. The Peltier coolers 40 are attached to the gloves 18 and the socks 20 so that they are positioned proximate to inner thumb surfaces, palmer sides of metacarpophalangeal joints, lower sides of metatarsophalangeal joints, and lower sides of big toes upon donning of the socks 20 and the gloves 18.

The present invention also anticipates the plurality of Peltier coolers 40 comprising Peltier coolers 40 that are positioned over the dorsum of forearms, posteriors of arms, lateral shoulders, inner lower shins, medial inner thighs, and lateral upper hips upon donning of the pants 22 and the long sleeved shirt 24. Additionally, the present invention anticipates the plurality of Peltier coolers 40 comprising Peltier coolers 40 that are selectively attachable to the user's abdomen, lower back, and face.

The vibrators 42 are attached the gloves 18, the socks 20, the pants 22, and the long sleeved shirt 24 and are configured for one or both of eliciting a vibratory sensation and a reflex response. The vibrators 42 have a vibratory range of 122 to 134 Hz. The plurality of vibrators 42 comprises a plurality of sensing inducers 44, the sensing inducers 44 of which are attached the gloves 18 and the socks 20 so that they are positioned proximate to distal bony prominences of the hands and feet upon donning of the gloves 18 and the socks 20, respectively. The sensing inducers 44 simulate running fork examination tools, which are well known to those skilled in the prior art of examination tools. The tuning fork test is a classical clinical examination tool in general neurology for diagnosis of Diabetic Peripheral Neuropathy, entrapment neuropathy, carpal tunnel, radiculopathy, fall risk, and pre-/post-operative nerve release. The user is asked to state when they start to feel the vibration and when they do not feel it anymore. Common anatomical sites for such tests are the first distal interphalangeal joint of the hand and the feet bilateral, bilateral lateral malleoli.

The plurality of vibrators 42 also comprises a plurality of reflex inducers 46, the reflex inducers 46 of which are attached to the long sleeved shirt 24 and the pants 22 so that they are positioned proximate to muscle tendons, thereby enabling testing of deep tendon reflexes. The present invention anticipates the reflex inducers 46 being attached to the long sleeved shirt 24 and the pants 22 so as to elicit reflexes from biceps, triceps, brachioradialis, knees, and ankles.

The present invention also anticipates additional sensors 28 and additional stimulators 30, as well as alternative positioning of the sensors 28 and the stimulators 30. For example, an additional blood pressure cuff 32 could be attached to the pants 22 proximate to ankle of the user and, in combination with the blood pressure cuff 32 attached to the long sleeved shirt 24, provide a means of determining an ankle-brachial pressure index for assessing a risk of peripheral arterial disease for the user.

The relay module 14 is attached to a respective one of the garments 12, such as to the long sleeved shirt 24, as shown in FIG. 2, although the relay module 14 also could be attached to the pants 22. The present invention also anticipates the relay module 14 being one of a plurality of relay module 14, with each relay module 14 being attached to a respective one of the plurality of garments 12 so that each garment 12 is equipped with a relay module 14.

The relay module 14 comprises a relay housing 48, which defines an interior space 50. A relay battery 52, a relay transceiver 54, and a relay microprocessor 56 are attached to the relay housing 48 and are positioned in the interior space 50, with the latter being operationally engaged to the relay battery 52, the relay transceiver 54, and the relay transceiver 54. The relay battery 52 is rechargeable. A relay port 58 attached to the relay housing 48 and is operationally engaged to the relay battery 52. The relay port 58 is configured for insertion of a plug 60 of a charging cord 62 to operationally engage the relay battery 52 to a source of electrical current to charge the relay battery 52.

A plurality of connectors 64 is attached to the plurality of garments 12, with respective connectors 64 being operationally engaged to the relay module 14. The connectors 64 are selectively mutually attachable, upon donning of the garments 12, so that the sensors 28 and the stimulators 30 are operationally engaged to the relay module 14. The plurality of connectors 64 is operationally engaged to cables 66 that are configured to carry both power and signals, such as, but not limited to, Universal Serial Bus cables, triaxial cables, category 5 cables, or the like. The connectors 64 are anticipated to comprise Universal Serial Bus couplers, Molex couplers, or the like.

The user interface device 16 is in wireless communication with the relay module 14 and a healthcare provider's electronic device 68. The user interface device 16 thus is configured to communicate commands, biological parameters, and biological responses between the relay module 14 and the healthcare provider's electronic device 68, allowing the healthcare provider to evaluate the user and to formulate a diagnosis.

The user interface device 16 comprises one or both of a controller 70, as shown in FIG. 6, and programming code 72, which is positioned on an electronic device of the user 74 enabling the electronic device 74 to formulate a diagnosis based on the biological parameters, the biological responses, and the strength and function of the hand. The programming code 72 on the electronic device of the user 74 also would allow the healthcare provider to selectively actuate the sensors 28 and stimulators 30 by means of the relay transceiver 54.

The controller 70 comprises a control housing 76, which define an internal space 78. A control battery 80, a control transceiver 82, a receiver 84, and a control microprocessor 86 are attached to the control housing 76 and are positioned in the internal space 78. The control microprocessor 86 is operationally engaged to the control battery 80, the control transceiver 82, and the receiver 84. The control transceiver 82 thus is configured to relay commands from, and to transmit the biological parameters and the biological responses to, the healthcare provider's electronic device 68. The receiver 84 is Global Positioning System enabled and thus is configured to receive positional coordinates of the controller 70, which correspond to positioning of the user.

The control battery 80 is rechargeable. A control port 88 is attached to the control housing 76 and is operationally engaged to the control battery 80. The control port 88 is configured for insertion of the plug 60 of the charging cord 62 to operationally engage the control battery 80 to a source of electrical current to charge the control battery 80.

As shown in FIG. 6, a handle 90, which is attached to and which extends from the control housing 76, is configured to be grasped in a hand of the user. A dynamometer 92 is attached to the control housing 76, between the handle 90 and the control housing 76, and is operationally engaged to the control microprocessor 86. The handle 90 and the dynamometer 92 are configured to be gripped and squeezed in the hand of the user to measure strength and function of the hand.

Software 94 is positioned on the control microprocessor 86 enabling the control microprocessor 86 to formulate a diagnosis based on the biological parameters, the biological responses, and the strength and function of the hand. The present invention also anticipates the controller 70 being in wireless communication with the electronic device of the user 74 so that the electronic device of the user 74 links the controller 70 to the healthcare provider's electronic device 68.

A control panel 96, a display 98, a speaker 100, and a microphone 102 are attached to the control housing 76 and are operationally engaged to the control microprocessor 86. The control microprocessor 86 is enabled to selectively actuate the display 98 to present one or more of a command from the healthcare provider, a biological parameter, a biological response, a dynamometer reading, and a diagnosis. The control microprocessor 86 also is enabled to selectively actuate the microphone 102 and the speaker 100 to enable two way communication between the user and the healthcare provider.

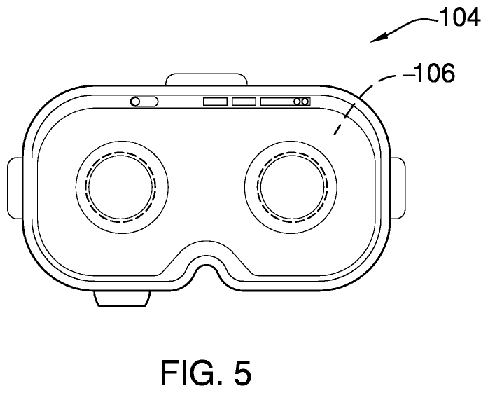

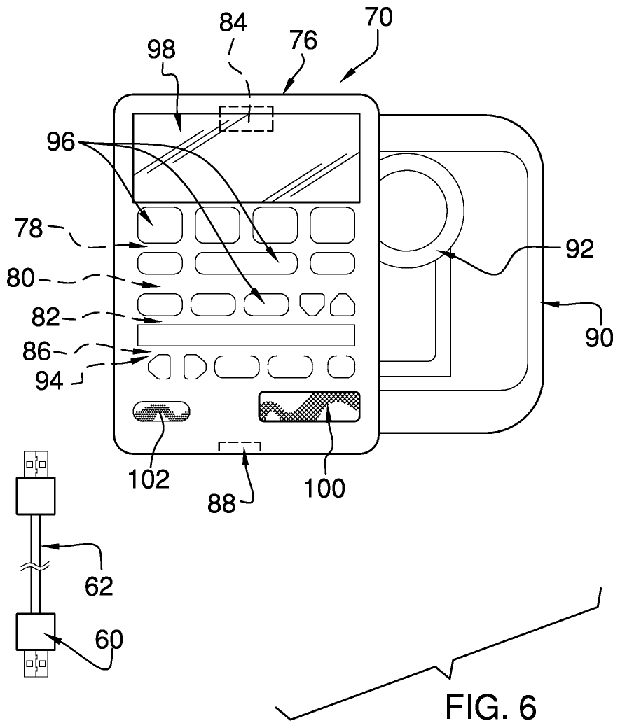

The remotely interfaced medical screening assembly 10 also may comprise a headset 104, as shown in FIG. 5, which is configured to be donned by a user and to display 98 an image, such as a virtual reality image, to the user. The headset 104 comprises a set transceiver 106 and thus is configured for wireless communication with the user interface device 16 or the electronic device of the user 74. The headset 104 is configured to enable virtual visitation with the healthcare provider, assessments of eye movement range and direction, pupil size, cognition testing, and rehabilitation exercises.

The remotely interfaced medical screening assembly 10 also may comprise a plurality of weights 108, the weights 108 of which are configured for selective attachment to respective garments 12, thereby enabling calculation of muscle strength of the user. The weights 108 may be attached to the respective garments 12 by means of straps, or the like. For testing strength of muscles in the arms of the user, the weights 108 may comprise hand grips 110, as shown in FIG. 1.

In use, the remotely interfaced medical screening assembly 10 enables a method for remotely screening a user for a medical condition 112. The method 112 comprises a provision step 114, which entails providing a remotely interfaced medical screening assembly 10, according to the specification above. A first preparatory step 116 of the method 112 is donning the plurality of garments 12. A second preparatory step 118 of the method 112 is mutually attaching the connectors 64 so that the sensors 28 and the stimulators 30 are operationally engaged to the relay module 14. A third preparatory step 120 of the method 112 is initiating wireless communication between the user interface device 16 and the healthcare provider's electronic device 68. A first use step 122 of the method 112 is allowing the sensors 28 to assess the biological parameters of the user. A second use step 124 of the method 112 is allowing the stimulators 30 to elicit the biological responses from the user. A third use step 126 of the method 112 is following any commands communicated by the healthcare provider.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A remotely interfaced medical screening assembly comprising:
a plurality of garments, each garment of the plurality of garments being configured for being donned by the user, such that the garment substantially covers a respective body part of the user;
a plurality of sensors, each sensor being attached to a respective garment of the plurality of garments, each sensor of the plurality of sensors being configured for assessing a respective biological parameter of the user, such that the plurality of sensors is configured for assessing a variety of biological parameters of the user;
a plurality of stimulators, each stimulator of the plurality of stimulators being attached to a respective garment of the plurality of garments, each stimulator of the plurality of stimulators being configured for eliciting a respective biological response from the user, such that the plurality of stimulators is configured for eliciting a variety of biological responses from the user;
a relay module attached to a respective one of the plurality of garments, the relay module comprising a relay transceiver;
a plurality of connectors attached to the plurality of garments, respective connectors of the plurality of connectors being operationally engaged to the relay module, the connectors of plurality of connectors being selectively mutually attachable upon donning of the plurality of garments, such that the plurality of sensors and the plurality of stimulators are operationally engaged to the relay module; and
a user interface device in wireless communication with the relay module and a healthcare provider's electronic device, wherein the user interface device is configured for communicating commands, biological parameters, and biological responses between the relay module and the healthcare provider's electronic device.

2. The remotely interfaced medical screening assembly of claim 1, wherein the plurality of garments comprises a pair of gloves, a pair of socks, pants, and a long sleeved shirt.

3. The remotely interfaced medical screening assembly of claim 2, wherein the long sleeved shirt comprises a zipper.

4. The remotely interfaced medical screening assembly of claim 2, wherein the plurality of sensors comprises:
a blood pressure cuff attached to a respective sleeve of the long sleeved shirt and being configured for measuring a blood pressure of the user;
a heart monitoring device attached to the long sleeved shirt and being configured for monitoring cardiac events;
a plurality of first motion sensors attached to the long sleeved shirt, such that the first motion sensors of the plurality of first motion sensors are positioned proximate to each shoulder, each elbow, and each wrist of the user upon donning of the long sleeved shirt, wherein the plurality of first motion sensors is configured for assessing range and direction of movement for upper limbs of the user; and
a plurality of second motion sensors attached to the pants, such that the second motion sensors of the plurality of second motion sensors are positioned proximate to each hip, each knee, and each ankle of the user upon donning of the pants, wherein the plurality of second motion sensors is configured for assessing range and direction of movement for lower limbs of the user.

5. The remotely interfaced medical screening assembly of claim 2, wherein the plurality of stimulators comprises:
a plurality of Peltier coolers attached to the gloves of the pair of gloves and the socks of the pair of socks, such that the Peltier coolers of the plurality of Peltier coolers are positioned proximate to inner thumb surfaces, palmer sides of metacarpophalangeal joints, lower sides of metatarsophalangeal joints, and lower sides of big toes upon donning of the pair of socks and the pair of gloves; and
a plurality of vibrators attached the gloves of the pair of gloves, the socks of the pair of socks, the pants, and the long sleeved shirt, such that the vibrators of the plurality of vibrators are configured for one or both of eliciting a vibratory sensation and a reflex response.

6. The remotely interfaced medical screening assembly of claim 5, wherein the vibrators of the plurality of vibrators have a vibratory range of 122 to 134 Hz.

7. The remotely interfaced medical screening assembly of claim 5, wherein the plurality of vibrators comprises:
a plurality of sensing inducers attached the gloves of the pair of gloves and the socks of the pair of socks, such that the sensing inducers of the plurality of sensing inducers are positioned proximate to distal bony prominences of the hands and feet upon donning of the pair of gloves and the pair of socks, respectively; and
a plurality of reflex inducers attached to the long sleeved shirt and the pants, such that the reflex inducers of plurality of reflex inducers are positioned proximate to muscle tendons for testing deep tendon reflexes.

8. The remotely interfaced medical screening assembly of claim 2, wherein the relay module is attached to the long sleeved shirt.

9. The remotely interfaced medical screening assembly of claim 1, wherein the relay module comprises:
a relay housing defining an interior space;
a relay battery attached to the relay housing and positioned in the interior space; and
a relay microprocessor attached to the relay housing and positioned in the interior space, the relay microprocessor being operationally engaged to the relay battery and the relay transceiver.

10. The remotely interfaced medical screening assembly of claim 9, further including:
the relay battery being rechargeable; and
a relay port attached to the relay housing and operationally engaged to the relay battery, wherein the relay port is configured for insertion of a plug of a charging cord for operationally engaging the relay battery to a source of electrical current for charging the relay battery.

11. The remotely interfaced medical screening assembly of claim 1, wherein the user interface device comprises one or both of:
a controller comprising:
a control housing defining an internal space,
a control battery attached to the control housing and positioned in the internal space,
a control transceiver attached to the control housing and positioned in the internal space,
a control microprocessor attached to the control housing and positioned in the internal space, the control microprocessor being operationally engaged to the control battery and the control transceiver, such that the control transceiver is configured for relaying commands from, and for transmitting the biological parameters and the biological responses to, the healthcare provider's electronic device,
a handle attached to and extending from the control housing, wherein the handle is configured for grasping in a hand of the user,
a dynamometer attached to the control housing between the handle and the control housing, the dynamometer being operationally engaged to the control microprocessor, wherein the handle and the dynamometer are configured for gripping and squeezing in the hand of the user for measuring strength and function of the hand,
software positioned on the control microprocessor enabling the control microprocessor for formulating a diagnosis based on the biological parameters, the biological responses, and the strength and function of the hand, and
a control panel attached to the control housing and operationally engaged to the control microprocessor; and
programming code positioned on an electronic device of the user enabling the electronic device for formulating a diagnosis based on the biological parameters, the biological responses, and the strength and function of the hand.

12. The remotely interfaced medical screening assembly of claim 11, further including:
a display attached to the control housing and operationally engaged to the control microprocessor, such that the control microprocessor is enabled for selectively actuating the display for presenting one or more of a command from a healthcare provider, a biological parameter, a biological response, a dynamometer reading, and a diagnosis;
a speaker attached to the control housing and operationally engaged to the control microprocessor; and
a microphone attached to the control housing and operationally engaged to the control microprocessor, such that the control microprocessor is enabled for selectively actuating the microphone and the speaker for enabling two way communication between the user and the healthcare provider.

13. The remotely interfaced medical screening assembly of claim 11, further including a receiver attached to the control housing, positioned in the internal space, and operationally engaged to the control microprocessor, the receiver being Global Positioning System enabled, wherein the receiver is configured for receiving positional coordinates of the controller.

14. The remotely interfaced medical screening assembly of claim 11, further including:
the control battery being rechargeable; and
a control port attached to the control housing and operationally engaged to the control battery, wherein the control port is configured for insertion of a plug of a charging cord for operationally engaging the control battery to a source of electrical current for charging the control battery.

15. The remotely interfaced medical screening assembly of claim 1, further including a headset configured for being donned by a user and for displaying an image to the user, the headset comprising a set transceiver, such that the headset is configured for wireless communication with the user interface device, wherein the headset is configured for enabling virtual visitation with the healthcare provider, assessments of eye movement range and direction, pupil size, cognition testing, and rehabilitation exercises.

16. The remotely interfaced medical screening assembly of claim 1, further including a plurality of weights, the weights of the plurality of weights being configured for selective attachment to respective garments of the plurality of garments for calculating muscle strength of the user.

17. A remotely interfaced medical screening assembly comprising:
a plurality of garments, each garment of the plurality of garments being configured for being donned by the user, such that the garment substantially covers a respective body part of the user, the plurality of garments comprising a pair of gloves, a pair of socks, pants, and a long sleeved shirt, the long sleeved shirt comprising a zipper;
a plurality of sensors, each sensor being attached to a respective garment of the plurality of garments, each sensor of the plurality of sensors being configured for assessing a respective biological parameter of the user, such that the plurality of sensors is configured for assessing a variety of biological parameters of the user, the plurality of sensors comprising:
a blood pressure cuff attached to a respective sleeve of the long sleeved shirt and being configured for measuring a blood pressure of the user,
a heart monitoring device attached to the long sleeved shirt and being configured for monitoring cardiac events,
a plurality of first motion sensors attached to the long sleeved shirt, such that the first motion sensors of the plurality of first motion sensors are positioned proximate to each shoulder, each elbow, and each wrist of the user upon donning of the long sleeved shirt, wherein the plurality of first motion sensors is configured for assessing range and direction of movement for upper limbs of the user, and
a plurality of second motion sensors attached to the pants, such that the second motion sensors of the plurality of second motion sensors are positioned proximate to each hip, each knee, and each ankle of the user upon donning of the pants, wherein the plurality of second motion sensors is configured for assessing range and direction of movement for lower limbs of the user,
a plurality of stimulators, each stimulator of the plurality of stimulators being attached to a respective garment of the plurality of garments, each stimulator of the plurality of stimulators being configured for eliciting a respective biological response from the user, such that the plurality of stimulators is configured for eliciting a variety of biological responses from the user, the plurality of stimulators comprising:
a plurality of Peltier coolers attached to the gloves of the pair of gloves and the socks of the pair of socks, such that the Peltier coolers of the plurality of Peltier coolers are positioned proximate to inner thumb surfaces, palmer sides of metacarpophalangeal joints, lower sides of metatarsophalangeal joints, and lower sides of big toes upon donning of the pair of socks and the pair of gloves, and
a plurality of vibrators attached the gloves of the pair of gloves, the socks of the pair of socks, the pants, and the long sleeved shirt, such that the vibrators of the plurality of vibrators are configured for one or both of eliciting a vibratory sensation and a reflex response, the vibrators of the plurality of vibrators having a vibratory range of 122 to 134 Hz, the plurality of vibrators comprising:
a plurality of sensing inducers attached the gloves of the pair of gloves and the socks of the pair of socks, such that the sensing inducers of the plurality of sensing inducers are positioned proximate to distal bony prominences of the hands and feet upon donning of the pair of gloves and the pair of socks, respectively, and
a plurality of reflex inducers attached to the long sleeved shirt and the pants, such that the reflex inducers of plurality of reflex inducers are positioned proximate to muscle tendons for testing deep tendon reflexes;
a relay module attached to a respective one of the plurality of garments, the relay module comprising a relay transceiver, the relay module being attached to the long sleeved shirt, the relay module comprising:
a relay housing defining an interior space,
a relay battery attached to the relay housing and positioned in the interior space, the relay battery being rechargeable,
a relay microprocessor attached to the relay housing and positioned in the interior space, the relay microprocessor being operationally engaged to the relay battery and the relay transceiver, and
a relay port attached to the relay housing and operationally engaged to the relay battery, wherein the relay port is configured for insertion of a plug of a charging cord for operationally engaging the relay battery to a source of electrical current for charging the relay battery;
a plurality of connectors attached to the plurality of garments, respective connectors of the plurality of connectors being operationally engaged to the relay module, the connectors of plurality of connectors being selectively mutually attachable upon donning of the plurality of garments, such that the plurality of sensors and the plurality of stimulators are operationally engaged to the relay module;
a user interface device in wireless communication with the relay module and a healthcare provider's electronic device, wherein the user interface device is configured for communicating commands, biological parameters, and biological responses between the relay module and the healthcare provider's electronic device, the user interface device comprising one or both of:
a controller comprising:
a control housing defining an internal space,
a control battery attached to the control housing and positioned in the internal space, the control battery being rechargeable,
a control transceiver attached to the control housing and positioned in the internal space,
a control microprocessor attached to the control housing and positioned in the internal space, the control microprocessor being operationally engaged to the control battery and the control transceiver, such that the control transceiver is configured for relaying commands from, and for transmitting the biological parameters and the biological responses to, the healthcare provider's electronic device,
a control port attached to the control housing and operationally engaged to the control battery, wherein the control port is configured for insertion of the plug of the charging cord for operationally engaging the control battery to a source of electrical current for charging the control battery,
a handle attached to and extending from the control housing, wherein the handle is configured for grasping in a hand of the user,
a dynamometer attached to the control housing between the handle and the control housing, the dynamometer being operationally engaged to the control microprocessor, wherein the handle and the dynamometer are configured for gripping and squeezing in the hand of the user for measuring strength and function of the hand,
software positioned on the control microprocessor enabling the control microprocessor for formulating a diagnosis based on the biological parameters, the biological responses, and the strength and function of the hand,
a display attached to the control housing and operationally engaged to the control microprocessor, such that the control microprocessor is enabled for selectively actuating the display for presenting one or more of a command from a healthcare provider, a biological parameter, a biological response, a dynamometer reading, and a diagnosis,
a speaker attached to the control housing and operationally engaged to the control microprocessor,
a microphone attached to the control housing and operationally engaged to the control microprocessor, such that the control microprocessor is enabled for selectively actuating the microphone and the speaker for enabling two way communication between the user and the healthcare provider,
a receiver attached to the control housing, positioned in the internal space, and operationally engaged to the control microprocessor, the receiver being Global Positioning System enabled, wherein the receiver is configured for receiving positional coordinates of the controller, and
a control panel attached to the control housing and operationally engaged to the control microprocessor, and
programming code positioned on an electronic device of the user enabling the electronic device for formulating a diagnosis based on the biological parameters, the biological responses, and the strength and function of the hand;
a headset configured for being donned by a user and for displaying an image to the user, the headset comprising a set transceiver, such that the headset is configured for wireless communication with the user interface device, wherein the headset is configured for enabling virtual visitation with the healthcare provider, assessments of eye movement range and direction, pupil size, cognition testing, and rehabilitation exercises; and
a plurality of weights, the weights of the plurality of weights being configured for selective attachment to respective garments of the plurality of garments for calculating muscle strength of the user.

18. A method for remotely screening a user for a medical condition comprising the steps of:
providing a remotely interfaced medical screening assembly comprising:
a plurality of garments, each garment of the plurality of garments being configured for being donned by the user, such that the garment substantially covers a respective body part of the user,
a plurality of sensors, each sensor being attached to a respective garment of the plurality of garments, each sensor of the plurality of sensors being configured for assessing a respective biological parameter of the user, such that the plurality of sensors is configured for assessing a variety of biological parameters of the user,
a plurality of stimulators, each stimulator of the plurality of stimulators being attached to a respective garment of the plurality of garments, each stimulator of the plurality of stimulators being configured for eliciting a respective biological response from the user, such that the plurality of stimulators is configured for eliciting a variety of biological responses from the user,
a relay module attached to a respective one of the plurality of garments, the relay module comprising a relay transceiver,
a plurality of connectors attached to the plurality of garments, respective connectors of the plurality of connectors being operationally engaged to the relay module, the connectors of plurality of connectors being selectively mutually attachable upon donning of the plurality of garments, such that the plurality of sensors and the plurality of stimulators are operationally engaged to the relay module, and
a user interface device in wireless communication with the relay module and a healthcare provider's electronic device, wherein the user interface device is configured for communicating commands, biological parameters, and biological responses between the relay module and the healthcare provider's electronic device;
donning the plurality of garments;
mutually attaching the connectors of the plurality of connectors so that the plurality of sensors and the plurality of stimulators are operationally engaged to the relay module;
initiating wireless communication between the user interface device and the healthcare provider's electronic device;
allowing the plurality of sensors to assess the biological parameters of the user;
allowing the plurality of stimulators to elicit the biological responses from the user; and
following any commands communicated by the healthcare provider.
